(19) United States
(12) Patent Application Publication
(10) Pub. No.: US 2024/0336495 A1
(43) Pub. Date: Oct. 10, 2024

(54) ADSORPTION METHOD FOR NITRILOTRIACETIC ACID (NTA) REMOVAL FROM EDTA CONTAINING CHELANT SOLUTIONS

(71) Applicant: Dow Global Technologies LLC, Midland, MI (US)

(72) Inventors: Deboleena Chakraborty, Midland, MI (US); Ronald R. Peterson, Midland, MI (US); Reza Hagh Panah, Midland, MI (US); Erich Molitor, Midland, MI (US); Mark A. Robertson, Gonzales, LA (US)

(21) Appl. No.: 18/577,434

(22) PCT Filed: Sep. 19, 2022
(86) PCT No.: PCT/US2022/043933

§ 371 Date: Jan. 8, 2024

Related U.S. Application Data
(60) Provisional application No. 63/245,991, filed on Sep. 20, 2021.

(51) Int. Cl. C02F 1/28; B01J 20/20; C02F 101/38; C07C 227/40

(52) U.S. Cl. CPC C02F 1/283; B01J 20/20; C07C 227/40; C02F 2101/38

(57) ABSTRACT

The present invention provides simple methods for treating aqueous chelating agent compositions containing one or more nitrilotriacetic acid (NTA) salts with one or more adsorbents, such as activated carbon, two or more activated carbon adsorbent in sequential treatments, to remove the NTA without removing more than 5 wt. %, or more than 3 wt. % or, preferably, more than 1 wt. % of the chelating agent in the aqueous chelating agent compositions. The methods comprise treating aqueous chelating agent compositions having a pH of from 7 to 12 and can be carried out at from 10 to 110 C. The methods can be used to treat a wide range of aqueous chelating agent compositions having, for example, from 700 to 6000 ppm of NTA, such as waste streams.

### FIELD

The present invention relates to methods for selectively separating nitrilotriacetic acid (NTA) salts from aqueous chelating agent compositions, such as manufacturing streams and wastewater streams, comprising contacting the aqueous compositions at a pH of from 7 to 12 with one or more adsorbent. More particularly, it relates to methods for selectively separating nitrilotriacetic acid (NTA) salts from aqueous chelating agent compositions comprising contacting the aqueous compositions with granular adsorbents, for example, having a sieve particle size or from 0.3 to 4 mm, preferably, activated carbons, or, more preferably, activated carbons from coconut, wood or hydrocarbon raw materials or, even more preferably, two or more thereof, particularly, treating in a sequential fashion with two or more thereof.

### INTRODUCTION

Known methods for making chelating agents, such as tetra sodium ethylenediamine tetraacetic acid (EDTA), include the addition of glycolonitrile (GN) (2-hydroxyacetonitrile) to ethylenediamine in the presence of aqueous sodium hydroxide, followed by hydrolysis under basic conditions to generate a polycarboxylate, also referred to as the Bersworth method. The hydrolysis reaction generates ammonia, which in turn reacts with the glycolonitrile, eventually leading to nitrilotriacetic acid (NTA) salts, a suspected carcinogen, as a by-product. It would be desirable to control the level of NTA to limit it to lower than 0.1 wt. %, based on the total weight of the aqueous chelating agent product.

A possible approach for NTA removal from aqueous chelating agent compositions could comprise separating them using an ion-exchange resin. However, ion-exchange resin separation would hamper the selective removal efficiency of NTA or its salts from an EDTA containing compositions regardless of whether they are aqueous products or internal streams. Stability constants of metal chelates of EDTA containing solutions are higher than that of NTA or its salts, and so one would expect that more of the EDTA than NTA salt would bind to the ion exchange resin, and in a more predictable way.

In a prior publication, Colloids and Surfaces A: Physicochem. Eng. Aspects 317 (2008), at pages 344-35, K. A. Krishnan has documented the adsorption of nitrilotriacetic acid onto activated carbon prepared by steam pyrolysis of sawdust as sorbent. Krishnan found that steam pyrolysed sawdust activated carbon (SDAC) was found to be more effective than commercial activated carbon (CAC) for the adsorption of NTA from aqueous solutions and wastewaters. Krishnan found the NTA removal process to be highly pH dependent, with best results obtained at an adsorbent pH of 5.0. Krishnan thus discloses a mechanism of adsorption for the removal of NTA comprising ligand exchange reaction between coordinated OH groups on the activated carbon surface and NTA (NTA²⁻ ions) in solution. Further, Krishnan discloses that removal efficiency decreases with an increase in the initial activated carbon concentration; therefore, kinetic adsorption equilibrium in the sorbent-solution interface was best explained by Langmuir adsorption isotherm, wherein adsorption sites become saturated with absorbate (NTA) molecules. The theory was that, adsorption being a surface phenomenon, smaller adsorbent sizes offered comparatively large surface area and hence higher adsorption occurs at equilibrium. However, Krishnan fails to disclose selective removal of NTA from chelating agent solutions; and the materials in Krishnan would not be expected to selectively remove NTA from an EDTA or chelating agent aqueous composition. Thus, there remains a need to selectively control NTA content in aqueous chelating agent compositions, removing NTA and not chelating agent.

In accordance with the present invention, the present inventors have solved the problem of providing a method for selectively separating nitrilotriacetic acid salts from aqueous chelating agent streams or compositions while removing less than 1 wt. % of the chelating agent from the compositions.

### SUMMARY

In accordance with the present invention, a method for selective removal of one or more nitrilotriacetic acid (NTA) salts from an aqueous chelating agent composition, such as ethylene diamine tetraacetate (EDTA) or its salts, or any chelating agent made using the Bersworth method of addition of glycolonitrile (GN) (2-hydroxyacetonitrile) to a polyalkylene polyamione, such as ethylenediamine, comprises treating the aqueous chelating agent composition at a pH of from 7 to 12 or, preferably, from 7.4 to 12, with one or more adsorbents, preferably, one or more granular adsorbents while retaining 95 wt. % or more, or, 97 wt. % or more or, preferably, 99 wt. % or more of the chelating agent in the aqueous chelating agent compositions. The aqueous chelating agent compositions treated may comprise from 1 to 60 wt. % or, preferably, from 10 to 45 wt. %, based on the total weight of the aqueous composition, of one or more chelating agents. Preferably, the one or more adsorbents comprise one or more activated carbons, or, more preferably, a combination of two or more activated carbons. The total amount of adsorbent may range from 0.5 to 12 wt. %, or, preferably, from less than 1 to 7.5 wt. %, based on the total weight of the aqueous chelating agent compositions. In the aqueous chelating agent compositions in accordance with the present invention, the content of NTA or its salts before the treating may range from 700 to 6500 ppm, such as 700 ppm or more, or 6000 ppm or less or, preferably, 750 ppm or more or, preferably, 3000 ppm or less.

Preferably, the activated carbon adsorbent comprises activated carbon having an iodine number as determined in accordance with ASTM D4607 of 600 or higher, or, preferably, 800 or higher or, more preferably, 900 or higher, or, even more preferably, 100 or higher. The activated carbon may come from a hydrocarbon, such as bituminous coal or lignite coal; wood; peat; or a nut shell, such as coconut, or, preferably, two or more of these. Preferably, the activated carbon is not acid washed prior to treating the aqueous chelating agent compositions.

Preferably, the method comprises treating the aqueous chelating agent composition containing NTA or its salts with an adsorbent, such as an activated carbon adsorbent, followed by removing the adsorbent from the aqueous chelating agent composition, and then treating the aqueous chelating agent composition a second time with an adsorbent, such as an activated carbon adsorbent. More preferably, to insure color body removal from the aqueous chelating agent compositions, the method of treating the aqueous chelating agent composition a second time with an adsorbent comprises treating with an activated carbon from a source other than wood, such as coconut.

### DETAILED DESCRIPTION OF THE INVENTION

In accordance with the present invention, methods of treating aqueous compositions of one or more chelating agents with adsorbents at a pH of from 7 to 12 provide highly selective nitrilotriacetic acid (NTA) salt removal and separation of the NTA salt from various aqueous chelating agent composition streams. The inventors have demonstrated experimentally a selective removal of NTA while retaining 99 wt. % or more or, for example, 99.5 wt. % or more of the chelating agent. In particular, treating any of an aqueous chelating agent composition having, for example, 40 wt. % solids or a waste stream comprising from 1 to 20 wt. % or, for example, from 10 to 20 wt. % of chelating agents via aqueous bulk methods enable selective removal of NTA salt from many kinds of chelating agents made from addition of glycolonitrile (GN) to ethylenediamine in the presence of aqueous sodium hydroxide, including ethylene diamine tetraacetic acid (EDTA), diethylene triamine pentaacetic acid (DTPA), diethylene triamine (DETA), hydroxyethyl ethylenediamine triacetic acid (EDTA), or any other chelating agent synthesized using the Bersworth method. In addition, the methods in accordance with the present invention enable the removal of cyanide or cyanide functional group (CN—) containing color bodies formed from the making of the aqueous chelating agent compositions in the presence of a base. The methods can be used to treat aqueous compositions having a wide range of initial concentrations of NTA or its salts, such as from 500 to over 10,000 ppm. For example, the present inventors have found that the inventive adsorbent treating methods enable an improved selective removal of NTA or its salts and separation thereof from aqueous chelating agent compositions including manufacturing streams, waste streams, reuse and recycle streams. For example, the methods of treating with one or more adsorbents can selectively remove NTA or its salts from various process or waste streams, that comprise of ethylene diamine tetraacetic acid (EDTA) chelating agents or their salts. Thus, the methods of the present invention find use in reducing waste and enable recycling of aqueous process stream compositions, including multiple or blended process streams having low concentrations of, for example, less than 1000 ppm of NTA or its salts. And the methods find use in batch as well as continuous manufacturing methods. Preferably, the one or more adsorbents comprises activated carbon, or a combination of two or more different activated carbons

The singular forms “a,” “an,” and “the” include plural referents unless the context clearly dictates otherwise. Unless defined otherwise, the terms used herein have the same meaning as is commonly understood by one skilled in the art.

Unless otherwise indicated, any term containing parentheses refers, alternatively, to the whole term as if no parentheses were present and the same term without that contained in the parentheses, and combinations of each alternative. Thus, the term “(meth)acrylate” encompasses, in the alternative, methacrylate, or acrylate, or mixtures thereof.

The endpoints of all ranges directed to the same component or property are inclusive of the endpoint and are independently combinable. Thus, for example, a disclosed range of from 700 to 6500 ppm of NTA or its salts, such as 700 ppm or more, or 6000 ppm or less or, preferably, 750 ppm or more or, preferably, 3000 ppm or less would include all of from 700 to 6500 ppm of NTA or its salts, or from 700 ppm to 6000 ppm, or from 700 to 750 ppm, or, from 700 to 3000 ppm, or, preferably, from 750 to 3000 ppm or, from 750 ppm to 6000 ppm, or from 750 to 6500 ppm, or, from 3000 to 6000 ppm or from 3000 to 6500 ppm of NTA or its salts.

Unless otherwise indicated, conditions of temperature and pressure are room temperature (23° C.) and standard pressure (101.3 kPa, also referred to as “ambient conditions”. And, unless otherwise indicated, all conditions include a relative humidity (RH) of 50%.

As used herein the term “aqueous” means that the continuous phase or medium is water and from 0 to 10 wt. %, based on the weight of the medium, of water-miscible compound(s) or molecules. Preferably, “aqueous” means water.

As used herein, the term “ASTM” refers to publications of ASTM International, West Conshohocken, PA.

As used herein, the term “sieve particle size” of a material refers to a particle size as determined by sieving the material through successively smaller size mesh sieves until at least 10 wt. % of the material is retained on a given sieve and recording the size of the sieve that is one sieve size larger than the first sieve which retains at least 10 wt. % of the material.

As used herein, the phrase “total solids”, “solids” or “as solids” refers to total amounts of any or all of the non-volatile ingredients or materials present in a given composition, including chelating agents, salts, adsorbents, reactants for making chelating agents, acids, polymers, inorganic materials, and other non-volatile materials and additives, such as caustic, regales of their physical state. Water, ammonia and volatile solvents are not considered solids.

The methods in accordance with the present invention comprise treating an aqueous chelating agent composition with one or more adsorbents, such as a granular adsorbent. The methods enable selective NTA salt removal from a wide variety of aqueous chelating agent compositions, including manufacturing products, intermediates and waste streams. Such aqueous chelating agent compositions can have a wide range of NTA or NTA salt concentration, such as from 700 ppm to over 6000 ppm. The methods may comprise of aqueous bulk methods wherein the aqueous chelating agent compositions have a varied solids and NTA or NTA salt content, for example, up to and above 50 wt. % solids, for example, from 5 to 40 wt. % solids, including waste streams at from 10 to 20 wt. % solids. The treating can be carried out in a conventional continuous or semi-continuous manner. Continuous treating may comprise, for example, eluting the aqueous chelating agent composition, such as through an elongated column or a series of elongated columns comprising the adsorbent affixed within them or placed upstream of a filter or grate. For example, the composition can be eluted upward through a column containing the adsorbent within it and having a downstream end located above an upstream end. Semi-continuous treating may comprise, for example, continually agitating, rotating or shaking an enclosed container or batch comprising the adsorbent and the aqueous chelating agent composition.

In accordance with the methods of the present invention, the aqueous chelating agent compositions have a pH of from 7 to 12 or, preferably, from 7.4 to 12 or, more preferably, from 7.4 to 11.7. The pH of the aqueous chelating agent compositions may be adjusted with a hard base, such as alkali or caustic, preferably, sodium or potassium hydroxide. Accordingly, the chelating agents treated in accordance with the present invention may include, for example, any alkali metal salt of ethylene diamine tetraacetic acid (EDTA), such as tetrasodium EDTA or trisodium EDTA or ED3A, symmetrical/unsymmetrical ethylene diamine diacetates, respectively SEDDA and UEDDA, referred to collectively as disodium EDTA, sodium ethylene diamine monoacetate (EDMA), or their mixtures. Further, the chelating agents treated in in accordance with the present invention may include any alkali metal salt of any of diethylene triamine pentaacetic acid (DTPA), diethylene triamine (DETA), or amine acid salts, such as, for example, bis(carboxymethyl)glutamate salts (GLDA, CAS 51981-21-6) or sodium N,N-bis(carboxymethyl) alanine (MGDA, CAS 164462-16-2. Examples of aqueous chelating agent compositions treated may include, for example, pentasodium DTPA, tetrasodium DTPA, trisodium DTPA, disodium DTPA, pentasodium DETA, tetrasodium DETA, trisodium DETA, disodium DETA, as well as mixtures of two or more of any alkali metal salts of DETA, DTPA, or EDTA.

In accordance with the methods of the present invention, the treating temperature at which the aqueous chelating agent compositions may be treated with the one or more absorbents may range from 10 to 110° C., or, for example, from 10 to 100° C., or, from 10 to 40° C., such as at room or ambient temperature. Thus, the methods in accordance with the present invention are economical.

Suitable amounts of the one or more adsorbents in total in the treating may range, for example, from 0.5 to 12 wt. %, or, preferably, from less than 1 to 7.5 wt. %, based on the total weight of the aqueous chelating agent compositions.

The methods in accordance with the present invention comprise treating the aqueous chelating agent compositions continuously, such as while pumping the aqueous chelating agent compositions through a column comprising part of a stream of the aqueous chelating agent composition, or, semi-continuously, or, for example, in a batch process for a period of from 30 minutes to 30 hours, such as from 18 to 24 hours. Preferably, the methods comprise treating the aqueous chelating agent compositions in a sequential fashion, such as by treating with one or more adsorbents, such as one or more granular absorbents, preferably, one or more activated carbons, for a first period or in a first phase and then treating with one or more activated carbons or, preferably, from coconut or a hydrocarbon for a second period or in a second phase. Each of the first and second period may range from 4 to 30 hours, such as from 18 to 24 hours; and each of the first and second phase may comprise a column comprising part of a stream of the aqueous chelating agent composition in a continuous process.

Various adsorbents are suitable for use in accordance with the treating methods of the present invention, including activated carbons, alumina, magnesium oxide, polymeric gel adsorbents or beads, such as those comprising supports for ion exchange resins like polystyrene or acrylamide beads, and clays, for example, bentonite. The various adsorbents may be granular or powdered, wherein powdered means a sieve particle size of under 0.5 mm. Examples of suitable adsorbents may include, for example, activated carbon from hydrocarbons, such as coal, including bituminous coal, or lignite coal; however, virtually any organic material that is high in carbon can be made into a suitable activated carbon adsorbent.

Granular adsorbents having a sieve particle size of the one or more adsorbents may range from 0.3 to 4 mm or, preferably, from 0.5 to 3 mm, or, preferably, from 0.6 to 1.7 mm are suitable for use in the treating of the present invention.

Preferred are activated carbons, such as those from coal, wood, peat, coconut and bamboo, and mixtures of two or more thereof, such as a blend of any activated carbon from coal with any of an activated carbon from any of wood, peat, coconut and bamboo or a blend of any activated carbon from wood or peat with any of an activated carbon from any of coal, coconut and bamboo.

Pore sizes in the adsorbent may range widely and can be, for example, greater than 50 nanometres. Suitable adsorbents may be macroporous or microporous.

Commercially available activated carbons from coal or coconut may include, for example, under the names Calgon (Calgon Corp, Pittsburgh, PA), DARCO or NORRIT (both to Sigma Aldrich, St. Louis) activated carbons; or activated carbon from coconut that is acid washed (OLC 12×30, Calgon) or not-acid washed (OLC AW 12×40, Calgon); or activated carbon from wood (ACTICARBONE BGE or ACTICARBONE BGX (Calgon Carbon Corp.); macroporous polymeric gel adsorbents (PUROSORB PAD550, PUROSORB PAD900 or PUROSORB PAD400, all to Calgon); polymer bead supports for ion exchange resins (PUROLITE MN200 (Calgon); and magnesium oxide (MAGNESOL XL, The Dallas Group of America, Jeffersonville, IN).

Preferably, the one or more adsorbents comprises an activated carbon or, more preferably, activated carbons from coconut, wood, peat or hydrocarbon raw materials or, even more preferably, two or more thereof.

The preferred activated carbon adsorbent has a different morphology depending on its source. The preferred activated carbon adsorbent from wood or peat generally has a microporous structure. The preferred activated carbon adsorbent from coconut shell is mostly microporous and has a high adsorption energy for removing contaminants of low molecular weight. The preferred activated carbon adsorbent from coal (anthracite, bituminous, and lignite) is both microporous and microporous. More preferably, the activated carbon adsorbent from bituminous Coal has an Iodine number of at least 900 or, preferably, the activated carbon adsorbent from lignite coal has an Iodine number of at least 600 and is not acid washed. More preferably, the activated carbon adsorbent from wood has an iodine number as determined in accordance with ASTM D4607 of greater than 900 or, preferably, greater than 1000. Even more preferably, the activated carbon adsorbent from wood is not acid washed. More preferably, the activated carbon from coconut has an iodine number of greater than 900.

The one or more adsorbents suitable for use in the treating method of the present invention may be chosen from granular adsorbents or bead adsorbents. Preferably, to enable easier and safer handling in use, at least one of the one or more adsorbents comprises a granular adsorbent having a sieve particle size of from 0.5 to 2 mm, or preferably, from 0.6 to 1.7 mm, such as or, preferably, an activated carbon having a sieve particle size of from 0.6 to 1.7 mm. Beads may or may not have the same sieve particle size as granules and can range as small as a sieve particle size of 0.3 mm.

The methods of the present invention find use in treating any aqueous chelating agent compositions having the disclosed amount of NTA or its sales, including compositions from chelating agent manufacturing and from uses downstream of the manufacturing where the chelating agent has been used, for example, to remove ions from process chemicals and compositions requiring high purity, like food and drug compositions or formulations containing them.

### EXAMPLES

The following examples illustrate the present invention. Unless otherwise indicated, all parts and percentages are by weight and all temperatures are in ° C. and all preparations and test procedures are carried out at ambient conditions of room temperature (23° C.) and pressure (1 atm). In the examples and Tables 1, 2, and 3 that follow, the following abbreviations were used: AC: Activated Carbon; High NTA EDTA feed: NTA salt concentration varies from −3000 ppm to over 5000 ppm.

Test Methods: The following test methods to evaluate adsorbent treatment of various aqueous composition feeds were used in the examples that follow:

Liquid Chromatography (analytical): Samples of the indicated feed or treated feed composition were prepared by weighing out a ˜0.2-0.3 g sample in a tared glass vial, adding ˜1 ml water and 5 drops of 50% (w/w) NaOH, cooking at 100° C. for 10 min., and adding 10 ml water to form a fully salted aqueous composition. The total weight was recorded. In a smaller tared vial, a ˜0.2-0.3 g sample was weighed, followed, without re-zeroing, by adding 1.5 ml of a 0.2 M Cu(OAc)₂ solution+0.5 ml of glacial acetic acid and recording the total weight. The resulting prepared samples were subject to liquid chromatography through a Waters Adsorbosphere SAX 4.6×250 mm×6 μm chromatographic column.

Shake tests (batch treatment) for adsorbent screening: Unless otherwise indicated, an amount of 5 wt. % of the indicated adsorbent, based on the total weight of the aqueous feed to be treated, was loaded in an upright sealed cylindrical vial containing the aqueous feed. The samples were equilibrated for 20-24 hours while being rotated around a horizontal axis, at 30 rpm at room temperature (20-25° C.).

Batch filtration: At the end of the desired time, the liquid was filtered to remove the adsorbents using a syringe filter (0.45 mm pore size, PTFE filter). The filtrate was a clear liquid free of particles. The filtered treated aqueous feed as well as the initial feed were analyzed using LC and the results for individual adsorbents studied are included in Tables 1A, 1B, 1C and 1D, below.

Aqueous Feed: Various aqueous feed compositions were tested. Unless otherwise indicated, the aqueous feed for any test comprised ˜39-40 wt. % tetrasodium EDTA and nitrilotriacetic acid (NTA) salt ≥4500 ppm, as determined by liquid chromatography (LC), and had a pH of 11.6.

Multiple Stage Batch treatment: An aqueous feed comprising ˜29.4 wt. % EDTA and NTA ≥3400 ppm as determined by liquid chromatography (LC) and having a pH of 11.6, was treated with 5 wt. %, based on the total weight of the composition, of the indicated adsorbent for 20 to 24 hours and filtered using the syringe filter. A portion of the clear effluent was analyzed and a filtered (effluent) aqueous feed composition was re-treated with 5 wt. %, based on the total weight of the effluent composition, of the indicated granular activated carbon for 20 to 24 hours and analyzed via LC. As shown in Tables 2A and 2B, below, the results demonstrated a reduction in each of NTA salts, symmetrical ethylenediamine-N,N′-diacetic acid (SEDDA), ethylenediaminetriacetic acid (ED3A), with almost no reduction in ethylenediamine tetraacetic acid (EDTA). Meanwhile, as shown in Tables 2A and 2B, below, the multistage batch treatments removed 30 wt. % of the original NTA salts.

Iodine Number: Adsorbent capacity of a material, as determined in accordance with ASTM D4607.

Molasses Number: Mesopore content for color body removal from of a material. The higher the number, the more mesopore capacity a material has.

p: The pH of a given material was that as reported by the manufacturer in Tables 1A, 1B, 1C and 1D, below.

TABLE 1A
Coal and Peat Sourced Adsorbents

| | Example | | | | | |
|---|---|---|---|---|---|---|
| | 1 | 2 | 3 | 4 | 5 | 6 |
| | Adsorbent No. | | | | | |
| | 15 | 10 | 18 | 14 | 24 | 2 |
| Material ID | Activated | Activated | CENTAUR | Activated | Activated | Activated |
| | Carbon (SGL | Carbon (CAL | Activated | Carbon (BPL | Carbon | Carbon |
| | 20 × 30)¹ | 12 × 40¹) | Carbon¹ | 4 × 10)¹ | (AQUASORB | (DARCO)² |
| | | | | | 5000 12 × 40)¹ | |
| Morphology | Granular, 500 | Granular, 450 | Granular, 450 | Granular, 1.8 | Granular, 450 | granular, 450 |
| and Shape; pH | to 850 μm | μm to 1.5 mm | μm to 1.5 mm | to 5 mm | μm to 1.5 mm | to 850 μm |
| | (20-30 mesh), | (12-40 mesh), | (12-40 mesh), | (4-10 mesh), | (12-40 mesh), | (20-40 mesh), |
| | Iodine number = 900, | Iodine = 1000, | Iodine = 825, | Iodine = 1000 | Iodine number | Iodine Number: |
| | molasses = | molasses = | Catalytic activated | | 1100; NA | 600, 650 m2/g |
| | 200; NA | 230; NA | carbon; NA | | | surface area; 4.8 |
| Source | Bituminous | Bituminous | Bituminous | Bituminous | Coal | Lignite Coal |
| | Coal | Coal | Coal | Coal | | |
| % NTA | −44% | −39% | −33% | −32% | −32% | −31% |
| removed | | | | | | |
| Initial Feed | 1630 | 2600 | 1630 | 1630 | 2600 | 1630 |
| NTA | | | | | | |
| concentration | | | | | | |
| (ppm) | | | | | | |

| | Example | | | | |
|---|---|---|---|---|---|
| | 7 | 8 | 9 | 10 | 11 |
| | Adsorbent No. | | | | |
| | 16 | 3 | 8 | 10 | 13 |
| Material ID | PWA | Activated | Activated | Activated | Pulverized |
| | Activated | Charcoal de- | Carbon | Carbon (CAL | Coal¹ |
| | Carbon¹ | colorizing 100² | NORIT PK3-5² | 12 × 40)¹ | |
| Morphology | Powdered, | Powder, ~150 μm | Granular, 1.3 | Granular, 450 m | Granular, 1.5 |
| and Shape; pH | acid washed, | (100 mesh): | to 5 mm | to 1.5 mm | mm to 3.3 mm |
| | 315 to 500 μm | 6 to 9 | (4-14 mesh), | (12-40 mesh), | (6-12 mesh); |
| | (30-60 mesh), | | Iodine number: | Iodine = 1000, | NA |
| | Iodine number | | 700; NA | molasses = | |
| | 900, molasses | | | 230; NA | |
| | number 210; 5 to 8 | | | | |
| Source | Bituminous | Lignite | Peat | Bituminous | Bituminous |
| | Coal | Coal | | Coal | Coal |
| % NTA | −31% | −28% | −27% | −27% | −27% |
| removed | | | | | |
| Initial Feed | 1630 | 1630 | 1630 | 1630 | 1630 |
| NTA | | | | | |
| concentration | | | | | |
| (ppm) | | | | | |

* Denotes Comparative Example. NA: Not applicable; ND: Not Determinate; nm: not meaningful;
¹Calgon, Pittsburgh, PA;
²Sigma Aldrich, St Louis, MO.

TABLE 1B
Coal and Peat Sourced Adsorbents

| | Example | | | | | | |
|---|---|---|---|---|---|---|---|
| | 12 | 13 | 14 | 15 | 16 | 17 | 18 |
| | Adsorbent No. | | | | | | |
| | 31 | 9 | 23 | 11 | 12 | 28 | 1 |
| Material ID | Activated | Powdered | Activated | Activated | pulverized | Activated | Activated |
| | Carbon | Activated | Carbon, | Carbon, | Activated | Carbon | Carbon |
| | (DARCO | Carbon, BL | Acid washed | SGL | Carbon | (DARCO | (DARCO, |
| | 12 × 20)¹ | PULV¹ | CPGLF | 8 × 30¹ | (PCG-B | 12 × 40)¹ | NORIT |
| | | | 12 × 40¹ | | PULV)¹ | | N.V.)² |
| Morphology | Granular, 850 | Iodine number: | Granular, 450 | Granular, 500 | Powder; NA | Granular, 450 | Granular, 1.5 |
| and Shape; pH | μm to 1.5 mm | 1000, | μm to 1.5 mm | μm to 3.0 mm | | μm to 1.5 mm | to 3.3 mm |
| | (12-20 mesh); | powder 65-75% | (12-40 mesh), | (8-30 mesh), | | (12-40 mesh), | (4 to 12 mesh), |
| | NA | through | iodine = 950, | Iodine | | Iodine | Iodine Number: |
| | | 42 μm | molasses = 210; | number = 900, | | number = 600, | 600; 4.8 |
| | | (325 mesh); | 5 to 8 | molasses = | | molasses = 90; | |
| | | 6 to 8 | | 200; NA | | NA | |
| Source | Lignite | Bituminous | Bituminous | Bituminous | Bituminous | Lignite | Lignite |
| | Coal | Coal | Coal | Coal | Coal | Coal | Coal |
| % NTA | −25% | −22% | −22% | −20% | −20% | −20% | −19% |
| removed | | | | | | | |
| Initial Feed | 2600 | 1630 | 2600 | 1630 | 1630 | 2600 | 1630 |
| NTA | | | | | | | |
| concentration | | | | | | | |
| (ppm) | | | | | | | |

| | Example | | | | | |
|---|---|---|---|---|---|---|
| | 19 | 20 | 21 | 22 | 23 | 24 |
| | Adsorbent No. | | | | | |
| | 6 | 7 | 17 | 25 | 5 | 4 |
| Material ID | Activated | Activated | Activated | pulverized | Activated | Activated |
| | Carbon | Carbon | Charcoal | Activated | Charcoal² | Charcoal |
| | (NORIT RO | (NORIT | DARCO | Carbon (SGL | | (Puriss. |
| | 0.8)² | PK1-3)² | KB-G² | 8 × 30)¹ | | p.a.²) |
| Morphology | Macro- | Macro- | Powder, >45 | Granular, | untreated, | powder; |
| and Shape; pH | porous, 0.8 | porous, | μm; 2 to 3.5 | micro-porous, | granular, 315 | 6 to 9 |
| | mm pellets; | Granular, 1- | | 500 μm to 3.0 mm | to 850 μm | |
| | NA | 3 mm; NA | | (8-30 mesh), | (20-60 mesh); | |
| | | | | Iodine number | 6 to 9 | |
| | | | | 900, | | |
| | | | | Molasses = | | |
| | | | | 200; NA | | |
| Source | peat | peat | Lignite | Bituminous | Lignite | Lignite |
| | | | Coal | Coal | Coal | Coal |
| % NTA | −19% | −19% | −18% | −18% | nm | nm |
| removed | | | | | | |
| Initial Feed | 1630 | 1630 | 1630 | 2600 | ND | ND |
| NTA | | | | | | |
| concentration | | | | | | |
| (ppm) | | | | | | |

* Denotes Comparative Example. NA: Not applicable; ND: Not Determinate; nm: not meaningful;
¹Calgon, Pittsburgh, PA;
²Sigma Aldrich, St Louis, MO.

TABLE 1C
Coconut and Wood Sourced Adsorbents

| | Example | | | | |
|---|---|---|---|---|---|
| | 25 | 26 | 27 | 28 | 29 |
| | Adsorbent ID | | | | |
| | 30 | 29 | 26 | 27 | 37 |
| Adsorbent | Activated | Activated | Activated | Activated Carbon | Activated Carbon |
| Material ID | Carbon (OLC | Carbon | Carbon (OLC | (ACTICARBONE | (ACTICARBONE |
| | 12 × 30)¹ | (NORIT GCN | AW 12 × | BGE)¹ | BGX)¹ |
| | | 1240 Plus)² | 40)¹ | | |
| Morphology | Granular, | Microporous, | Microporous, | Macroporous, | Macroporous, |
| and Shape | microporous, | Iodine = | Iodine = | Iodine = | Iodine = |
| | 500 μm to | 1000; | 1050; | 1025, | 1025, |
| | 1.5 mm (12-30 | 5 to 8 | 8 | molasses >380; | molasses >330; |
| | mesh) | | | 5 | 5.5 |
| | Iodine = | | | | |
| | 900; NA | | | | |
| Source | Coconut | Acid Washed | Acid Washed | Wood | Wood |
| | | Coconut | Coconut | | |
| % NTA | −30% | −23% | −17% | −31% | −31% |
| removed | | | | | |
| Initial Feed | 2600 | 2600 | 2600 | 2600 | 3100 |
| NTA | | | | | |
| concentration | | | | | |
| (ppm) | | | | | |

* Denotes Comparative Example.
NA: Not applicable;
ND: Not Determinate;
nm: not meaningful;
¹Calgon, Pittsburgh, PA;
²Sigma Aldrich, St Louis, MO.

TABLE 1D
Other Adsorbents

| | Example | | | | | | | | |
|---|---|---|---|---|---|---|---|---|---|
| | 30 | 31 | 32 | 33 | 34 | 35 | 36 | 37 | 38 |
| | Absorbent ID | | | | | | | | |
| | 20 | 19 | 36 | 35 | 32 | 33 | 34 | 22 | 21 |
| Adsorbent | Alumina | Alumina, | Activated | Non- | Non- | Poly- | Non- | Ben- | Magnesium |
| Material | A-540 | Activated, | Alumina | functionalized | functionalized | acrylamide | functionalized | tonite | Oxide |
| ID | | basic | | Polymeric | Polymeric | (PUROSORB | Polymeric | | (MAGNESOL |
| | | (Brockmann | | Adsorbents | Adsorbents, | PAD550)¹ | Adsorbents | | XL)³ |
| | | I)² | | (PUROSORB | (PUROLITE | | (PUROSORB | | |
| | | | | PAD400)¹ | MN200)¹ | | PAD900)¹ | | |
| Morphology | 80 to 225 | Powder, | spherical, | beads, 700 | contains both | beads, 800 | beads, 850 | Powder; | — |
| and | μm (80- | 106 μm (150 | 1.3 to 3 mm | m2/g surface | micropores | m2/g surface | m2/g surface | 7 to | |
| Shape | 200 | mesh), 58 Å | (7 to 14 | area; NA | and | area; NA | area; NA | 10.5 | |
| | mesh); | pore size, 205 | mesh), | | macropores, | | | | |
| | NA | m2/g surface | surface area | | 1100 m2/g | | | | |
| | | area; 9.5 | 360 m2/g; | | surface area; | | | | |
| | | | NA | | NA | | | | |
| Source | Alumina | Alumina | Alumina | Polymeric | Polymeric | Polymeric | Polymeric | Clay | Earth |
| | | | | Adsorbents | Adsorbents | Adsorbents | Adsorbents | | |
| % NTA | −31% | −30% | −9% | −13% | −12% | −12% | −11% | −35% | −33% |
| removed | | | | | | | | | |
| Initial Feed | 1630 | 1630 | 2900 | 2900 | 2900 | 2900 | 2900 | 1630 | nm |
| NTA con- | | | | | | | | | |
| centration | | | | | | | | | |

* Denotes Comparative Example. NA: Not applicable; ND: Not Determinate; nm: not meaningful;
¹Calgon, Pittsburgh, PA;
²Sigma Aldrich, St Louis, MO;
³The Dallas Group of America, Jeffersonville, IN.

TABLE 2A
Concentrations of Multiple Stage Batch Selective
Removal using Granular Activated Carbon from
Coal of Example 2 on two consecutive days

| | Shake Test, | Shake Test, | |
|---|---|---|---|
| Analytes | Day 1 | Day 2 | Feed |
| SEDDA | 0.24% | 0.24% | 0.26% |
| ED3A | 0.36% | 0.35% | 0.52% |
| NTA | 0.26% | 0.24% | 0.34% |
| EDTA | 29.32% | 29.46% | 29.46% |

TABLE 2B
Multiple Stage Batch Selective Removal Results
using the Activated Carbon of Example 2

| | AC | | Eq. Conc. | NTA | NTA | % NTA |
|---|---|---|---|---|---|---|
| | loading | NTA | NTA | Conc | Absorbed | re- |
| Sample | (wt. %) | (wt. %) | (mg/L) | (mg) | (mg/g) | moved |
| Feed | 0.0% | 0.34% | | | | |
| Shake Test, | 4.9% | 0.26% | 2593.0 | 25.89 | 16.57 | 25% |
| Day 1 | | | | | | |
| Shake Test, | 4.9% | 0.24% | 2423.8 | 24.30 | 19.79 | 30% |
| Day 2 | | | | | | |

Selective Removal efficiency: As shown in Table 2A, above, the EDTA concentrate after 2 days of consecutive shake tests or batch treatment was only about 0.5% less than the initial concertation of the EDTA in the feed; meanwhile, the adsorbent removed 30 wt. % of total NTA salts. As shown in Table 3A, below, the batch methods of the present invention using mixed adsorbents selectively removed NTA salt from an aqueous composition having about 3200 ppm of NTA, while amounts of ED3A and SEDDA and EDTA in an aqueous feed treated with a 50:50 by mass mix of an activated carbon from wood at a pH of 5 and one from coal at various solid: liquid ratios. Additionally, the adsorbent blends effectively work at a low solid to NTA ratio.

TABLE 3A
Selective Removal Results Using Activated Carbon 50:50 Blends

| | | AC no. 37 | AC no 37 | AC no 37 | AC no 37 |
|---|---|---|---|---|---|
| | Feed | and no. 10 | and no 10 | and no 10 | and no. 10 |
| Analytes | (wt. %) | (1 wt. %) | (5 wt. %) | (10 wt. %) | (20 wt. %) |
| SEDDA | 0.25 | 0.24 | 0.12 | 0.12 | — |
| ED3A | 0.38 | 0.36 | 0.25 | 0.25 | — |
| NTA | 0.315 | 0.226 | 0.223 | 0.206 | — |
| EDTA | 29.35 | 29.39 | 29.49 | 29.42 | — |

TABLE 3B
Removal Efficiency in Blend Batch Treatments

| | | Eq. | | | |
|---|---|---|---|---|---|
| Load | | Conc. | NTA | | Solid:Liquid |
| AC no 37 and | NTA | NTA | Absorbed | % NTA | Ratio |
| AC no. 10 | (wt. %) | (mg/L) | (mg/g) | removed | (w/w) |
| Feed (0 wt. %) | 0.31% | | | | 0 |
| 1 wt. % | 0.23% | 2264.0 | 84.58 | 28% | 1.0% |
| 5 wt. % | 0.22% | 2226.9 | 17.57 | 29% | 5.2% |
| 10 wt. % | 0.21% | 2060.1 | 9.87 | 35% | 11.0% |
| 20 wt. % | — | — | — | — | 24.8% |

Adsorption Isotherms: Adsorption isotherms were generated using overnight shake tests for varying adsorbent: liquid ratios of the adsorbents. An anti-Langmuir isotherm behavior, showing greater relative removal at lower adsorbent concentrations was noted for the systems studied as depicted in Table 4. Adsorption speed was not increased with use of more activated carbon.

TABLE 4A
Activated Carbon Isotherms with Adsorbent no. 10

| | Adsorbent: | |
|---|---|---|
| Conc. Of | Granular¹ | |
| NTA (by LC) | Aqueous | |
| for Granular | Chelating | % NTA |
| Adsorbent | Agent | removed |
| Tested | Composition | by Granular |
| (mg/L) | Ratio | AC |
| 2949.6 | 1.0% | −16% |
| 2666.2 | 4.9% | −24% |
| 2447.4 | 5.1% | −31% |
| 2601.7 | 10.1% | −26% |
| 2553.1 | 20.2% | −28% |
| 2382.3 | 28.1% | −32% |
| 2393.1 | 34.2% | −32% |

¹Adsorbent no. 10 as granule (450 μm to 1.5 mm size).

TABLE 4B
Activated Carbon Isotherms with Adsorbent no. 10

| Conc. Of NTA | Adsorbent: Powder¹ | |
|---|---|---|
| (by LC) for | Aqueous Chelating | % NTA |
| Adsorbent | Agent Composition | removed by |
| Powder (mg/L) | Ratio | Powder AC |
| ~2900 | — | −7% |
| 2687.0 | 1.0% | −7% |
| 2505.6 | 5.3% | −13% |
| 2411.1 | 10.0% | −17% |
| 2420.6 | 20.6% | −16% |
| 2283.3 | 24.8% | −21% |

¹Adsorbent no. 10 as powder, particle size not measured.

As shown in Tables 4A and 4B, above, in shake tests adsorbent particle size distribution (PSD) was tested versus NTA removal efficiency for an NTA containing EDTA feed using granular and manually (mortar and pestle) crushed activated carbon from bituminous coal showed that lowering particle size did not improve the NTA removal efficiencies.

Removal Testing at Higher Temperature: An amount of ˜5 wt. % of the adsorbent 10 activated carbon (crushed and granular forms) were loaded in a sample vial containing high NTA Versene™ 100 chelating agent feed. A magnetic stir bar was added to the vial. The vial was sealed using an electrical tape. The samples were equilibrated at 50° C. in a heater block for 24 hours at the indicated rpm. As shown in Table 5, below, a temperature increase did not improve NTA removal efficiency.

TABLE 5
Effect of Temperature on Treatment Using Activated Carbon Adsorbent no. 10

| | | | | Eq. Conc. | NTA | |
|---|---|---|---|---|---|---|
| | Treating | % AC | NTA | NTA | Absorbed | % NTA |
| Event | Conditions | loading | (wt. %) | (mg/L) | (mg/g) | removed |
| Feed | | 0.0% | 0.29% | ~2900 | — | — |
| Granules RT | 24 hours @ 30 | 4.9% | 0.24% | 2447.4 | 8.69 | −15% |
| | rpm, 20 C. | | | | | |
| Granules | 24 hours @ 100 | 5.0% | 0.24% | 2437.9 | 8.62 | −16% |
| 50 C. | rpm, 50 C., | | | | | |
| Crushed RT | 24 hours @ 30 | 5.0% | 0.25% | 2505.6 | 733.0% | −13% |
| | rpm, 20 C. | | | | | |
| Crushed | 24 hours @ 100 | 4.9% | 0.24% | 2394.4 | 9.60 | −17% |
| 50 C. | rpm, 50 C., | | | | | |
| | Crushed | | | | | |

Varying aqueous composition pH: The pH of the high NTA (>˜3000 ppm) containing EDTA aqueous composition was adjusted in shake tests using hydrochloric acid. The resulting pH was measured using a calibrated pH meter. Three different feed streams were generated (pH 12, pH 10, pH 7, and pH 5). At pH 5, the solids crashed out of the solution, and hence were not considered for the study. The rest of the samples were treated and analyzed, as described above. As shown in Table 6, below, with a lower pH the NTA removal efficiency improves; however, as pH goes below 10 selectivity for not removing EDTA deteriorates.

TABLE 6
Effect of pH on NTA Removal Efficiency

| Activated Carbon | NTA | % NTA | % EDTA |
|---|---|---|---|
| Absorbent no. 10 | (wt %) | removed | Removed |
| pH 12 feed | 0.35% | | |
| pH 12 feed with | 0.27% | −24% | −5% |
| Absorbent 10 | | | |
| pH 10 Feed | 0.36% | | |
| ph 10 Feed with Absorbent | 0.236% | −34% | 0% |
| 10 | | | |
| pH 7 Feed | 0.349% | | |
| pH 7 feed with Cal 12 × 40 | 0.186% | −47% | 19% |

Varying NTA concentration: A high NTA EDTA feed was diluted using DI water resulting in varied starting NTA feed concentrations. A series of shake tests were conducted by mixing low and high NTA containing EDTA feeds at various proportions. The solid: liquid ratios for all these studies were ˜5 wt %. The samples were processed and analyzed under identical conditions and analyzed following the protocol, as described above as shown in Table 7, below, the lower the feed NTA concentration, the better the removal efficiency. Mixed adsorbent results are also depicted in Table 7. The inclusion of a bituminous coal sourced activated carbon, adsorbent no. 10 or a coconut sourced activated carbon, adsorbent no. 26 improved the efficiency of the wood sourced activated carbon, adsorbent no. 37 at NTA feed concentrations of 0.10 wt. % and 0.31 wt. %.

TABLE 6
Effect of NTA Concentration on Removal
Efficiency, Including Mixed Absorbents
Adsorbent

| No. 37 | | No. 37 and no. 10 | | No 37. and no. 26 | |
|---|---|---|---|---|---|
| Feed NTA | | Feed NTA | | Feed NTA | |
| conc | % NTA | conc | % NTA | conc | % NTA |
| (wt %) | reduction | (wt %) | reduction | (wt %) | reduction |
| 0.31% | 31% | 0.31% | 42% | 0.31% | 35% |
| 0.17% | 33% | 0.10% | 52% | 0.10% | 45% |
| 0.15% | 39% | 0.01% | 100% | 0.01% | 100% |
| 0.12% | 48% | | | | |
| 0.10% | 28% | | | | |
| 0.01% | 100% | | | | |

NTA removal efficiency was tested by mixing various NTA feeds containing various amounts of NTA (high, having ˜3100 ppm NTA and low, having ˜1900 ppm of NTA) and various amounts of adsorbents, including 50:50 (w/w) mixtures of adsorbents in shake tests, and the results are presented in Table 7, below. As shown in Table 7, The lower the amount of NTA in the starting feed, the more effective the treatment, with treatment of a low NTA feed removing as much and more than half the remaining NTA, whereas treatment of a higher NTA feed removed less and less of the NTA per 1 wt. % of adsorbent or mixed adsorbent. However, at only a 1 wt. % amount of adsorbent, the high NTA treatment was most effective, removing almost 30% of the NTA. Treatments were generally effective in feeds having as little as 800 ppm of NTA.

TABLE 7
Impact of NTA Concentration In Feed

| | | | Eq. | | |
|---|---|---|---|---|---|
| | | | Conc. | NTA | NTA |
| Feed and | solid:Liq | NTA | NTA | Conc | Absorbed |
| Adsorbent no. | ratio | (wt %) | (mg/L) | (mg) | (mg/g) |
| High NTA Feed | | 0.31% | 0.0 | 0.00 | 0.00 |
| High NTA + 1% of 50:50 | 1% | 0.23% | 2307.1 | 11.56 | 83.31 |
| no. 10 and no. 26 | | | | | |
| High NTA + 5% of 50:50 | 6% | 0.23% | 2332.5 | 11.26 | 14.67 |
| no. 10 and no. 26 | | | | | |
| High NTA + 10% of 50:50 | 11% | 0.24% | 2372.0 | 10.71 | 6.86 |
| no. 10 and no. 26 | | | | | |
| High NTA and 20% of 50:50 | 25% | 0.25% | 2490.3 | 10.03 | 2.64 |
| no. 10 and no. 26 | | | | | |
| 50:50 low and high NTA Feed | | 0.25% | 0.0 | 0.00 | 0.00 |
| 50:50 + 1% of 50:50 | 1% | 0.22% | 2206.6 | 21.91 | 29.27 |
| no. 10 and no. 26 | | | | | |
| 50:50 + 5% of 50:50 | 5% | 0.20% | 1950.1 | 18.53 | 10.82 |
| no. 10 and no. 26 | | | | | |
| 50:50 + 10% of 50:50 | 11% | 0.19% | 1917.4 | 17.26 | 5.38 |
| no. 10 and no. 26 | | | | | |
| 50:50 + 15% of 50:50 | 18% | 0.18% | 1840.5 | 15.64 | 3.80 |
| no. 10 and no. 26 | | | | | |
| 90:10 low and high NTA Feed | | 0.20% | 0.0 | 0.00 | 0.00 |
| 90:10 + 1% of 50:50 | 1% | 0.15% | 1482.1 | 14.92 | 54.08 |
| no. 10 and no. 26 | | | | | |
| 90:10 + 5% of 50:50 | 5% | 0.13% | 1304.2 | 12.39 | 13.39 |
| no. 10 and no. 26 | | | | | |
| 90:10 + 10% of 50:50 | 11% | 0.07% | 742.0 | 6.69 | 11.41 |
| no. 10 and no. 26 | | | | | |
| 90:10 + 15% of 50:50 | 18% | 0.07% | 654.7 | 5.57 | 7.66 |
| no. 10 and no. 26 | | | | | |
| Low NTA feed | | 0.09% | 926.3 | 0.00 | 0.00 |
| Low + 1% of 50:50 | 1% | 0.08% | 824.2 | 4.11 | 10.13 |
| no. 10 and no. 26 | | | | | |
| Low + 5% of 50:50 | 5% | 0.08% | 752.6 | 3.57 | 3.26 |
| no. 10 and no. 26 | | | | | |
| Low + 10% of 50:50 | 11% | 0.05% | 491.8 | 2.24 | 3.86 |
| no. 10 and no. 26 | | | | | |
| Low + 20% of 50:50 | 25% | 0.04% | 433.1 | 1.74 | 2.00 |
| no. 10 and no. 26 | | | | | |

Adsorbent PH variation Testing: The impact of adsorbent pH was studied using the low NTA ([INTA}˜ 900 ppm) VERSENE 100 HP stream (pH ˜11.6) and the results appear in Table 8, below from shake tests 5 wt. % adsorbent: feed ratio, 24 hours, 20° C. Wood sourced activated carbons like those sold as ACTICARBONE-BGX and ACTICARBONE-BG (Calgon) have a lower pH (˜5) and exhibited relatively better NTA removal efficiency for the same feed under the same test conditions as compared to coal or coconut based activated carbons. See Table 1C, above and Table 8, below. However, the effluents treated with wood sourced activated carbon contained color bodies primarily due to their high ash content. Pre-washing the carbon or staged treatment with either Cal 12×40 (Calgon) activated carbon from coal or OLC 12×30 (Calgon) activated carbon from coconut shell afterward was effective to remove color bodies from the treated effluent streams.

TABLE 8
Effect of Adsorbent pH

| Activated | % Reduction |
|---|---|
| Carbon | in NTA |
| No. 10 (Cal 12 × 40)¹ | 22% |
| No. 26 (OLC 12 × 30)¹ | 67% |
| (ACTICARBONE BGE)¹ | 74% |
| No. 37 (ACTICARBONE BGX)¹ | 100% |

¹Calgon.

Staged activated carbon treatments: As shown in Table 9, below, using two activated carbon shake treatments in sequence improved NTA removal efficiency from low NTA EDTA feeds having about 580 ppm (0.058 wt. %) of NTA and from high NTA EDTA feeds having about 4100 ppm (0.41 wt. %) of NTA. Specifically, staged treatments with each of mesoporous (micro and microporous) bituminous coal sourced activated carbon (Cal 12×40 or adsorbent no. 10), and microporous coconut shell sourced activated carbons (OLC 12×30 or adsorbent no. 26) helped improve NTA removal efficiency.

TABLE 9
Sequential Shake Test Results

| | Adsorbent | % NTA |
|---|---|---|
| | no | removed |
| Stage of treatment | | |
| of Low NTA feed | | |
| 1 | 10 | −6% |
| 2 | 10 | −100% |
| 1 | 10 | −20% |
| 2 | 26 | −81% |
| 1 | 26 | −67% |
| 2 | 10 | −100% |
| Stage of treatment | | |
| of high NTA feed | | |
| 1 | 10 | −30% |
| 2 | 10 | −32% |
| 1 | 10 | −32% |
| 2 | 26 | −38% |
| 1 | 26 | −31% |
| 2 | 10 | −43% |

Breakthrough Testing: In breakthrough studies, the indicated aqueous composition was fed at room temperature through a single pass 1.23m (4 ft) packed bed, 1.59 cm (0.625″) ID at a flow rate of 5 ml/min, maintaining the temperature to ±2° C. with a water jacket. A 184 kpa (12 psig) pressure increase was observed across the column. In a single adsorbent, high NTA feed test, 132.5 g of Absorbent no. 10 was loaded in the column. The column was not pre-wetted with water. A high NTA EDTA ([NTA} ˜4100 ppm) aqueous composition was passed through the column from bottom up. Samples were collected every 15 min in the first 2 hours and then every hour after that. Immediate breakthrough of the column was observed. However, as shown in Table 10A, below, a ˜44% reduction in NTA was maintained over 8 hours of continuous operation. Further breakthrough tested was carried out using a staged column of 66 g of Absorbent no. 26 was loaded first in a dry column followed by 73 g of Absorbent no. 10. A pre-mixed 90:10 low: high NTA EDTA aqueous composition was introduced to the dry column from the bottom at 5 ml/min feed rate. Immediate breakthrough was observed. However, as shown in Table 10B, below, a ˜20% reduction in NTA was maintained over 6 hours of continuous operation.

TABLE 10A
Single Adsorbent Breakthrough Test

| t | C_{NTA} | Feed NTA |
|---|---|---|
| (h) | (ppm) | (ppm) |
| 0 | 2671 | 4448 |
| 0.5 | 3279 | 4448 |
| 1 | 2330 | 4448 |
| 2 | 2288 | 4448 |
| 3 | 2832 | 4448 |
| 4 | 2794 | 4448 |
| 5 | 2187 | 4448 |
| 6 | 2163 | 4448 |
| 7 | 2153 | 4448 |
| 8 | 2129 | 4448 |

TABLE 10B
Staged Adsorbent Breakthrough Test

| t | C_{NTA} | Feed NTA | % reduction |
|---|---|---|---|
| (h) | (ppm) | (ppm) | (%) |
| 0 | 863 | 1040 | 17% |
| 0.25 | 841 | 1040 | 19% |
| 0.5 | 848 | 1040 | 18% |
| 0.75 | 915 | 1040 | 12% |
| 1 | 891 | 1040 | 14% |
| 1.25 | 914 | 1040 | 12% |
| 1.5 | 846 | 1040 | 19% |
| 1.75 | 825 | 1040 | 21% |
| 2 | 826 | 1040 | 21% |
| 3 | 805 | 1040 | 23% |
| 4 | 801 | 1040 | 23% |
| 5 | 791 | 1040 | 24% |
| 5.5 | 808 | 1040 | 22% |

1. A method for selective removal of one or more nitrilotriacetic acid (NTA) salts from an aqueous chelating agent composition comprises treating the aqueous chelating agent composition at a pH of from 7 to 12 with one or more adsorbents while retaining 95 wt. % or more of the chelating agent in the aqueous chelating agent compositions, wherein the aqueous composition comprises from 1 to 60 wt. %, based on the total weight of the aqueous composition, of one or more chelating agents.

2. The method as claimed in claim 1, wherein the pH of the aqueous chelating agent composition ranges from 7.4 to 12 and the amount of adsorbent ranges from 0.5 to 12 wt. %, based on the total weight of the aqueous chelating agent compositions.

3. The method as claimed in claim 1, wherein the aqueous chelating agent compositions comprise one or more NTA salts in an amount of from 700 to 6500 ppm before the treating.

4. The method as claimed in claim 1, wherein at least one of the adsorbents comprises an activated carbon.

5. The method as claimed in claim 4, wherein the adsorbent comprises a combination of two or more activated carbons.

6. The method as claimed in claim 4, wherein the activated carbon adsorbent comes from a hydrocarbon, wood, peat, a nut shell, or two or more of these.

7. The method as claimed in claim 4, wherein the activated carbon adsorbent has an iodine number as determined in accordance with ASTM D4607 of 600 or higher.

8. The method as claimed in claim 7, wherein the activated carbon adsorbent has an iodine number as determined in accordance with ASTM D4607 of 800 or higher.

9. The method as claimed in claim 1, further comprising removing the adsorbent from the aqueous chelating agent composition and then treating the aqueous chelating agent composition a second time with an adsorbent.

10. The method as claimed in claim 9, wherein the treating comprises treating the aqueous chelating agent composition with an activated carbon adsorbent and the treating a second time comprises treating with an activated carbon from a source other than wood.
